(19) United States
(12) Patent Application Publication
(10) Pub. No.: US 2004/0234605 A1
(43) Pub. Date: Nov. 25, 2004

(54) Antimicrobial adhesive system

(76) Inventors: David D. Cox, Woodbury, MN (US); Robert E. Lund, Eagan, MN (US); Leland W. Annett, Baytown, MN (US)

Correspondence Address: NAWROCKI, ROONEY & SIVERTSON, SUITE 401, BROADWAY PLACE EAST, 3433 BROADWAY STREET NORTHEAST, MINNEAPOLIS, MN, 554133009

(21) Appl. No.: 10/717,380

(22) Filed: Nov. 19, 2003

Related U.S. Application Data
(63) Continuation-in-part of application No. 10/644,049, filed on Aug. 19, 2003.
Application No. 10/644,049 is a continuation of application No. 10/202,232, filed on Jul. 24, 2002, now Pat. No. 6,607,746.
Application No. 10/202,232 is a continuation of application No. 09/836,764, filed on Apr. 17, 2001, now Pat. No. 6,503,531.
Application No. 09/836,764 is a continuation of application No. 09/185,456, filed on Nov. 3, 1998, now Pat. No. 6,216,699.
Application No. 09/185,456 is a continuation of application No. 08/662,850, filed on Jun. 12, 1996, now Pat. No. 5,829,442.

(51) Int. Cl. A61K 9/14

(52) U.S. Cl. 424/486

(57) ABSTRACT

An adhesive composition having dispersed therein a broad spectrum antimicrobial agent for use in medical applications, such as an adhesive for surgical drapes, wound dressings and tapes, is provided. The adhesive is composed of acrylic polymers, tackifiers and a preferred antimicrobial agent, diiodomethyl-p-tolylsulfone. The subject adhesive composition may be formulated as either an essentially solventless hot melt, or as a solvent based system wherein an emulsion of the antimicrobial agent and the removal of excess solvent is avoided.

This is a continuation-in-part of application Ser. No. 10/644,049 filed Aug. 19, 2003 which is a continuation of application Ser. No. 10/202,232 filed July. 24, 2002, now U.S. Pat. No. 6,607,746 issued on Aug. 19, 2003 which is a continuation of application Ser. No. 09/836,764 filed Apr. 17, 2001, now U.S. Pat. No. 6,503,531 issued on Jan. 7, 2003, which is a continuation of application Ser. No. 09/185,456 filed Nov. 3, 1998, now U.S. Pat. No. 6,216,699 issued on Apr. 17, 2001 which is a continuation of application Ser. No. 08/662,850 filed on Jun. 12, 1996, now U.S. Pat. No. 5,829,442 issued on Nov. 3, 1998.

### TECHNICAL FIELD

The present invention relates to a medical grade, antimicrobial-containing adhesive particularly suited for use in skin contact applications, such as with surgical drapes, tapes and wound dressings. More particularly, the subject adhesive system includes an acrylic polymer in conjunction with diiodomethyl-p-tolylsulfone antimicrobial agent.

### BACKGROUND OF THE INVENTION

It is recognized that numerous pathogens are present on human skin. Therefore, in a hospital environment, it is generally desired that the growth of disease-producing microorganisms be inhibited, and preferably that these microorganisms be destroyed so as to control patient infection and encourage wound healing. Under most circumstances, the bacteria of normal skin cannot cause wound infections, but in the presence of foreign materials or open wounds, the pathogenic potential of these bacteria appears to be considerably enhanced. Furthermore, the likelihood of bacterial contamination is at a peak immediately preceding, during, and following surgical procedures. Accordingly, to prevent contamination, it is imperative that the skin be effectively disinfected before a surgical incision is made and during the entire surgical procedure.

In response to such concerns, many topical antimicrobial agents have been developed. These agents typically are in the form of preoperative skin preps, surgical scrub tissues, washes, wound cleaners, lotions and ointments. A recognized limitation to such topical applications are a short effective delivery time. Microorganisms that may have survived the initial application of such a topical antimicrobial agent can act as a seed, causing the pathogen population in some instances to regenerate or rise to their initial levels. Thus, continuous application of an antimicrobial agent to the site is recognized as a means of inhibiting this increase in population.

It has been recognized that a continuous or longer lasting antimicrobial effect may be achieved by incorporating the antimicrobial agent into an adhesive layer or into a surgical incise drape material itself.

Berglund et al. (U.S. Pat. No. 4,310,509) disclose that it is known to incorporate biologically active agents into adhesive layers on a substrate to provide continuous application of such agent to the body. Disclosed examples of known adhesives containing antimicrobial agents include U.S. Pat. No. 2,137,169, wherein phenol, thymol, methanol, etc. are added to a starch adhesive; U.S. Pat. No. 3,249,109 where benzocaine was added to a tacky gelatin; U.S. Pat. No. 3,632,740 where a corticosteroid is added to an adhesive; U.S. Pat. No. 3,734,097 where a microencapsulated anti-neoplastic agent is added to an adhesive; U.S. Pat. No. 4,073,291 where Tretinoin is added to an adhesive; U.S. Pat. No. 3,769,071 where 5-fluorouracil is incorporated into an adhesive; and U.S. Pat. No. 3,896,789 where retinoic acid is incorporated into a pressure-sensitive adhesive tape. Berglund et al. further teach that the prior art attempts to include an antimicrobial agent in an adhesive did not include the use of a broad spectrum antimicrobial because such adhesives had been frustrated by uncontrollable release of the agent with accompanying skin irritation in some patients, along with failure to obtain sufficient antimicrobial activity.

Berglund et al. disclose a pressure sensitive adhesive composition which contains chlorhexidene, polyvinylpyrrolidone iodine or iodine which is applied onto a polymer sheet material, such as polyethylene or polyurethane, for use as a surgical drape. The disclosed drape is applied to the skin with the adhesive side contacting the skin so that the antimicrobial agent can be released from the adhesive to the wound area prior to and during incision. The process for making the adhesive disclosed by Berglund et al. involves forming an emulsifiable concentrate or an organic solution concentrate of a broad spectrum antimicrobial agent and mixing it into an adhesive, such that the broad spectrum antimicrobial is homogeneously dispersed as a separate phase throughout the adhesive medium. The homogenous dispersion is then spread or coated to a substantially uniform layer followed by drying of the wet layer in order to remove the solvents.

Rosso et al. (U.S. Pat. No. 4,323,557) disclose a drape incorporating a pressure sensitive adhesive utilizing n-vinylpyrrolidione residues in the polymer backbone. Iodine is complexed with these residues to provide an antimicrobial effect. Rosso et al. espouse the stability of the adhesive composition over the prior art compositions. By stable, Rosso et al. asserts that a composition coating of 11 grains per 24 sq. in. which is attached to a polyethylene sheet can be exposed to a temperature of 120° F. and a relative humidity of 9% for two weeks or to a dose of 2.5 megarads of gamma irradiation without substantial alteration of the physical appearance or of the chemical activity as tested by the starch test and microbiological activity as tested by the zone inhibition assay. The disclosure of Rosso et al. is incorporated herein by reference.

The process for forming the adhesive composition disclosed by Rosso et al. involves forming a pressure-sensitive adhesive and mixing into it an antimicrobial treating solution comprising iodine, an iodide, and a solvent. The resulting composition preferably contains n-vinylpyrrolidone in the backbone of the pressure-sensitive adhesive which serves to complex the iodine. Rosso et al. disclose that the composition may be either attached directly onto a flexible backing substrate or formed onto a release liner for later use. Once applied, the solvents are then evaporated by means known to the art, whereby an adhesive film is formed which is useable in or on tapes, drapes and other medical devices.

Mixon et al. (U.S. Pat. No. 5,069,907) disclose a surgical drape having incorporated therein a broad spectrum antimicrobial agent. The drape comprises a synthetic polymeric film or fabric having incorporated therethrough an amount of antimicrobial agent. The drape may optionally have an adhesive layer attached to one of its external surfaces, wherein the adhesive layer can have dispersed therethrough an antimicrobial agent. The preferred antimicrobial agent used is 5-chloro-2-(2,4-dichlorophenoxy)phenol. Suitable adhesives utilized include polyacrylate adhesives.

Mixon et al. disclose a large number of antimicrobial agents which were contemplated for use with the disclosed composition. These include metal salts, typical antibiotics, antibacterial agents such as chlorhexidine and its salts, quaternary ammonium compounds, iodophors such as povidone iodine, acridine compounds, biguanidine compounds, and a preferred antimicrobial agent 5-chloro-2-(2,4-dichlorophenoxy)phenol. Mixon et al. further disclose that these same antimicrobial agents, which they propose to utilize within the polymer composition for their surgical drape, can also be utilized in an adhesive composition. Mixon et al. further state that the antimicrobial agent can be directly applied to the surgical drape in solution as an aqueous dispersion, as a hot melt, or by a transfer process using known techniques, such as knife, roller-coating, or curtain-coating methods. The transfer process is disclosed as particularly preferred. In a transfer process, the adhesive emulsion, including water or a different solvent, optionally containing an antimicrobial agent, is spread onto a sheet of release paper and dried to remove the water or solvent. The surgical drape is then brought into contact with the adhesive and calendared to insure that the adhesive adheres to the drape. The surgical drape will then generally include a release sheet covering the adhesive, and the release sheet on which the adhesive is deposited can be used for that purpose, or that release sheet can be removed and replaced with another release sheet. In embodiments where the adhesive contains an antimicrobial agent, the mixture of adhesive and antimicrobial agent is dried after coating on the release sheet, and the antimicrobial agent remains dispersed in the adhesive.

Generally, presently known antimicrobial agents are limited in their ability to withstand heat during processing. The lack of heat stability of n-vinyl pyrrolodione iodine has limited the ability for drapes having this antimicrobial agent from being ethylene oxide sterilized under heat stress. Further, many of the antimicrobial compounds cannot be radiation sterilized. Thus, each prior art reference teaches that it is preferred to apply the antimicrobial adhesive in conjunction with a solvent followed by subsequent evaporation of the solvent.

Accordingly, the need exists for an adhesive composition having an antimicrobial agent dispersed therethrough which is heat stable, and solventless. Similarly, it is highly desirable and advantageous to provide an antimicrobial system requiring neither an emulsion of the antimicrobial agent, or the removal of excess solvent. Such composition or compositions would eliminate the need for use of solvents with their potential environmental effects and would eliminate the need for removing such solvent from the adhesive after application to the drapes. Furthermore, a particularly heat stabile formulation would allow the antimicrobial system to be applied in a hot melt process, while also allowing for ethylene oxide sterilization under heat stress or radiation sterilization.

### SUMMARY OF THE INVENTION

An adhesive composition having dispersed therein a broad spectrum antimicrobial agent for use in medical applications, such as an adhesive for surgical drapes, wound dressings and tapes is provided. The adhesive is composed of acrylic polymers, tackifiers and a preferred antimicrobial agent, diiodomethyl-p-tolylsulfone. The subject adhesive composition may be formulated as either an essentially solventless hot melt, or as a solvent based system wherein an emulsion of the antimicrobial agent and the removal of excess solvent is avoided.

The solventless adhesive composition formulation of the subject invention is essentially 100% solids, and heat stable, so that it may be applied in a hot melt process, while also being capable of ethylene oxide sterilization under heat stress without loss of effectiveness of the antimicrobial agent. Specifically, the adhesive is for skin-contact applications, for example, surgical drapes, tapes and wound dressings. The antimicrobial agent utilized is diiodomethyl-p-tolylsulfone with a preferred concentration of antimicrobial agents in the adhesive of about 0.1% to about 2% loading by weight.

The antimicrobial agent is homogeneously dispersed through the adhesive layer. Active antimicrobial molecules continually disassociate from the surface or leach out of the adhesive matrix over time, delivering biocidal activity at a distance from the adhesive surface. Applicants have conclusively demonstrated this property by zone of inhibition tests on a wide variety of infectious organisms. These tests conclusively showed that microbes were inhibited at a distance from the sample.

Adhesive compositions can incorporate acrylic or rubber based polymers to form the hot melt adhesive. A preferred composition includes a mixture of two acrylic polymers, one of which is a low molecular weight solid acrylic polymer, the other a medium molecular weight solid acrylic polymer, which are both designed for hot melt pressure-sensitive adhesive applications. A low molecular weight solid acrylic polymer is available from Schenectady International, Inc. as Product No. HRJ-4326, and a medium molecular weight solid acrylic polymer is also available from Schenectady International, Inc. under Product No. HRJ-10127. Tackifiers can also be added to the adhesive composition as is well known in the art.

The present adhesive composition is a hot melt adhesive. By hot melt adhesive, it is meant that the adhesive is essentially solventless or 100% solids and is processed in liquid form at elevated temperatures in the range of about 275° F. to 350° F. A preferred temperature range for compounding and coating the antimicrobial adhesive is 290° F. to 320° F. The antimicrobial containing adhesive is manufactured by heating the adhesive composition to about 250° F., including both a low molecular weight acrylic polymer and a medium molecular weight acrylic polymer along with any tackifiers to be utilized. The mixture is then heated to about 310° F. to about 315° F. and mixed until uniform with subsequent cooling to 290° F. to 295° F. at which point the diiodomethyl-p-tolylsulfone is added. The composition is mixed until uniform with subsequent packaging and cooling. The composition may then be hot melted and applied as needed by the user.

In a preferred application, the antimicrobial adhesive composition of the present invention is utilized to overly a polymeric substrate to form a surgical drape. The polymeric substrate is preferably a polyester or co-polyester sheet material which may have incorporated therein or coated on the side opposite the adhesive an antimicrobial agent.

The solvent based adhesive composition formulation of the subject invention is particularly advantageous as it does not require an emulsion of the antimicrobial agent, nor the removal of excess solvent, and generally includes an acrylic polymer, and an effective amount of diiodomethyl-p-tolylsufone dispersed throughout the acrylic polymer. The acrylic polymer suitably comprises a mixture of acrylic resin solutions, more particularly, self-curing and non self-curing acrylic resin solutions, the non self-curing acrylic resin solution preferably present in the mixture to a greater extent than the self-curing acrylic resin solution.

These and various other advantages and features of novelty which characterize the present invention are pointed out with particularity in the claims annexed hereto and forming a part hereof. However, for a better understanding of the invention, its advantages, and the objects obtained by its use, reference should be made to the accompanying descriptive matter in which there are illustrated and described preferred embodiments of the present invention.

### BRIEF DESCRIPTION OF THE DRAWINGS & TABLES

FIG. 1 is an enlarged, sectional illustration of a first embodiment of the present invention;

FIG. 2 is an enlarged, sectional illustration of a second embodiment of the present invention;

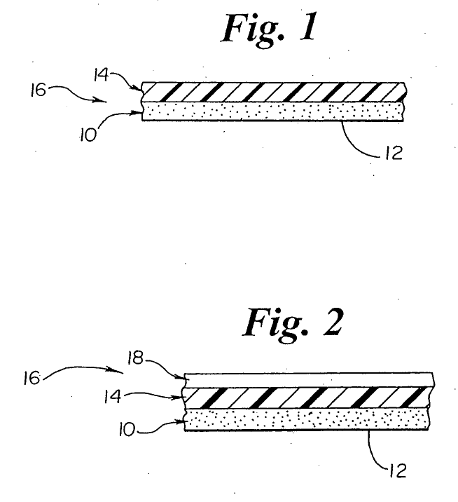

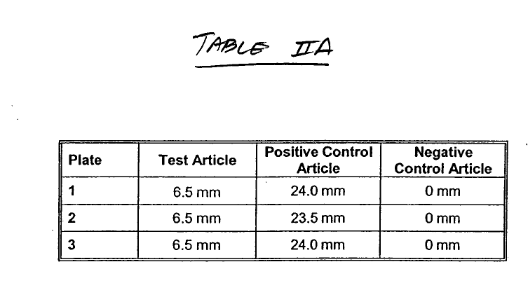

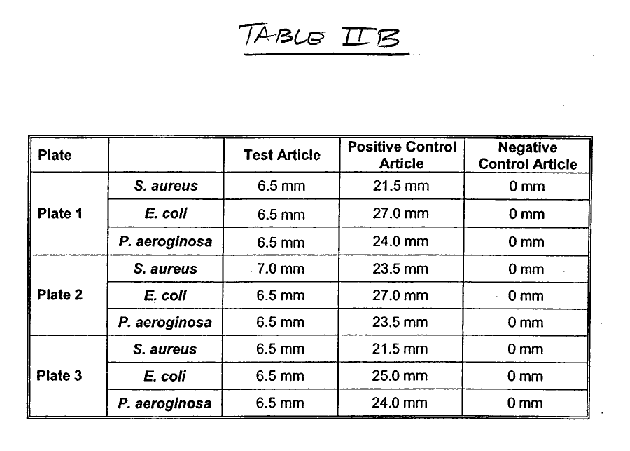

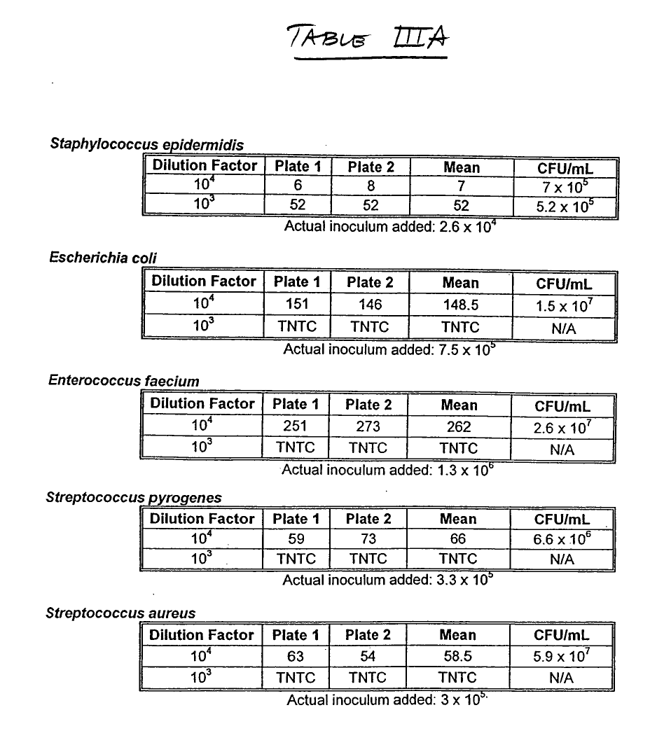

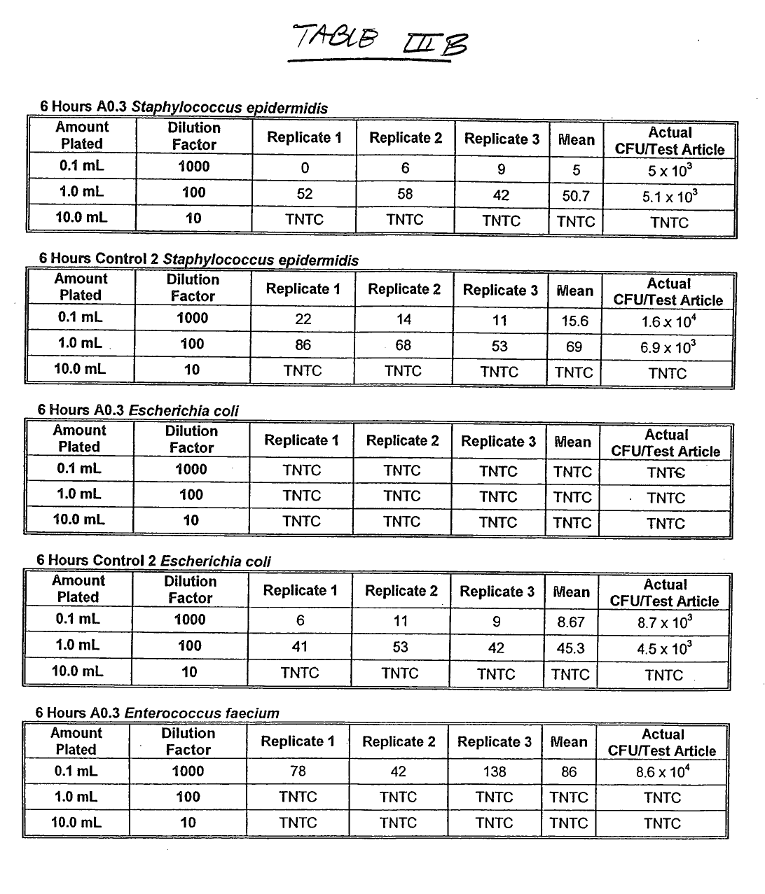

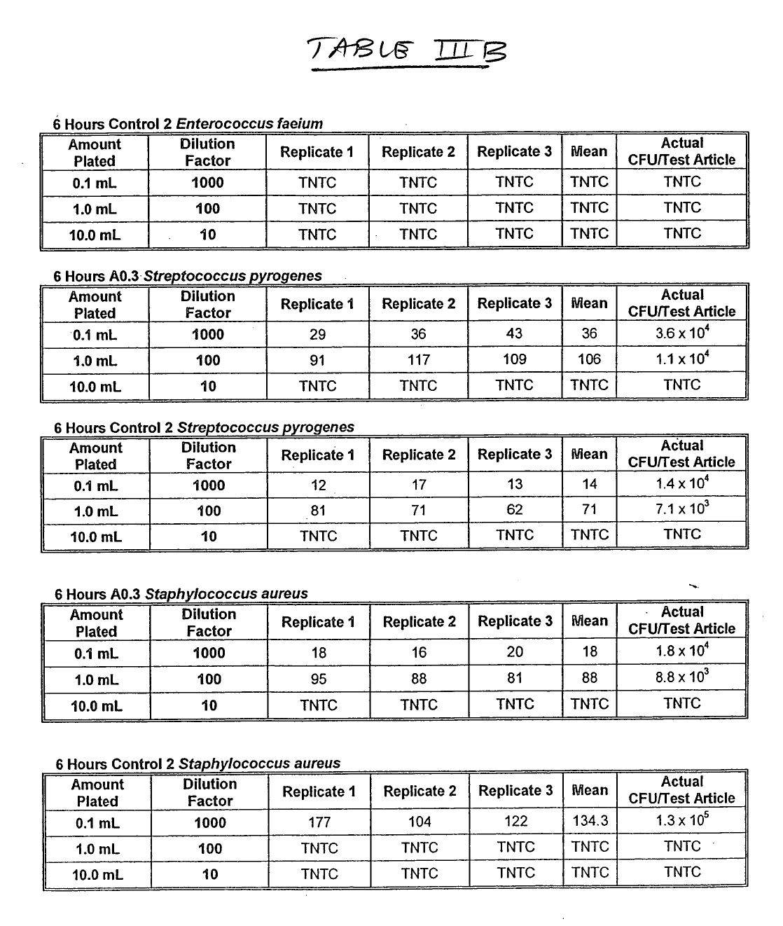

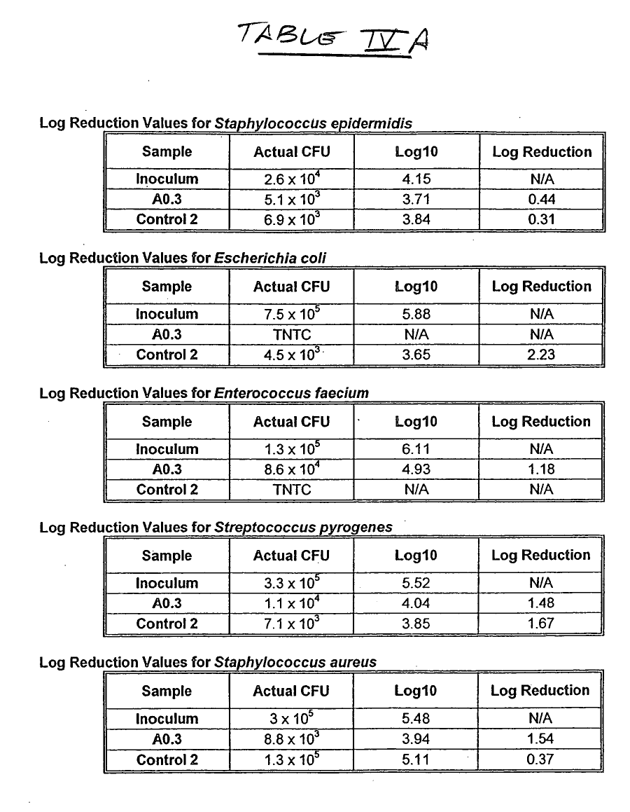

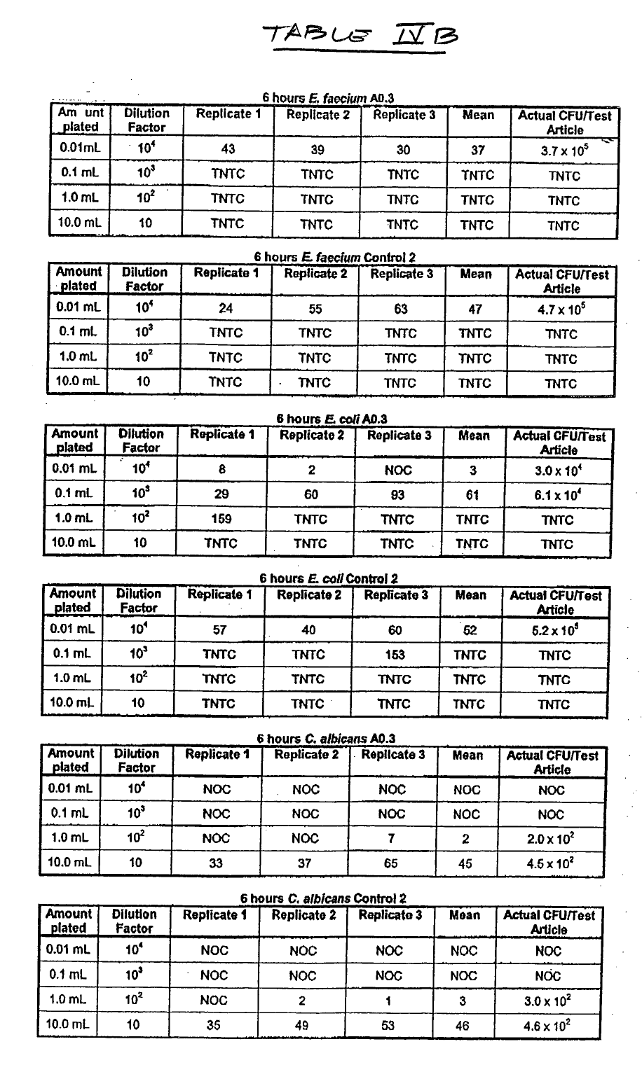

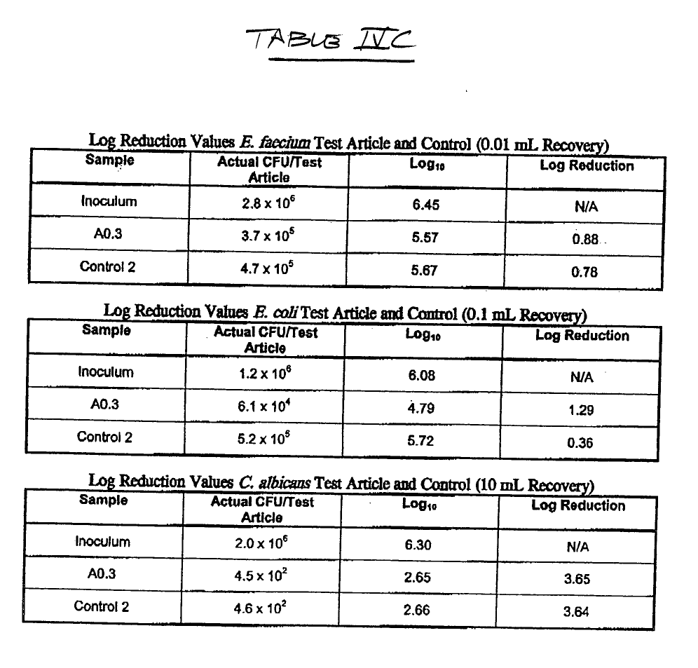

Tables I summarizes zone of inhibition test results for a solventless antimicrobial adhesive formulation for the subject invention;

Tables IIA & IIB summarize zone of inhibition test results for a solvent based antimicrobial adhesive solvent formulation for the subject invention;

Tables IIIA-IIIC summarize a first set of log reduction effectiveness test results for the solvent based antimicrobial adhesive solvent formulation as reported with respect to Tables IIA & IIB; and,

Tables IVA & IVB summarize a further or second set of log reduction effectiveness test results for the solvent based antimicrobial adhesive solvent formulation as reported with respect to Tables IIIA-IIIC.

### DETAILED DESCRIPTION OF THE PREFERRED EMBODIMENTS

As required, detailed embodiments of the present invention are disclosed herein. However, it is to be understood that the disclosed embodiments are merely exemplary of the present invention which may be embodied in various systems. Therefore, specific details disclosed herein are not to be interpreted as limiting, but rather as a basis for the claims and as a representative basis for teaching one skilled in the art to variously practice the present invention.

The present invention is an adhesive compound which incorporates an adhesive component together with a broad spectrum antimicrobial agent dispersed therethrough. The antimicrobial agent is homogeneously dispersed throughout the adhesive layer 10. Active antimicrobial molecules of the present composition disassociate from the surface or leach out of the adhesive matrix over time, delivering biocidal activity at a distance from the adhesive surface 12. Applicants have conclusively demonstrated by zone of inhibition tests on a wide variety of infections organisms the efficacy of the present composition. These tests showed that microbes were inhibited and killed at a distance from the sample as detailed in the attached experimental examples. In all embodiments, an adhesive composition is provided, the formulations thereof being characterized by an acrylic polymer and an effective amount of diiodomethyl-p-tolylsufone dispersed throughout the acrylic polymer.

The adhesive of the present invention is specifically suited for use in skin contact applications during and after medical procedures, for example; as an adhesive in surgical drapes 16, wound dressings and tapes. The adhesive composition is a hot melt adhesive. By hot melt adhesive, it is meant that the adhesive is essentially solventless or 100% solids and flowable at elevated temperatures for application to a substrate material 14, such as a surgical drape. The preferred adhesive composition incorporates acrylic polymers and added tackifiers to form a pressure-sensitive adhesive which is particularly suited for use in surgical procedures.

A preferred combination of acrylic polymers to form the adhesive composition includes the combination of a low molecular weight solid acrylic polymer and a medium molecular weight solid acrylic polymer in a ratio of about 1 to 4, respectively, to optimize the adhesion of the adhesive to skin, cohesion and resistance to cold flow. A low molecular acrylic polymer is a polymer having a molecular weight ranging from about 90,000 to about 120,000, while a medium molecular weight acrylic polymer has a molecular weight ranging from about 140,000 to about 160,000. Suitable low molecular weight solid acrylic polymers and medium molecular weight solid acrylic polymers are available from Schenectady International, Inc. under Product Nos. HRJ-4326 and HRJ-10127, respectively.

The adhesive component of the composition can also include tackifiers as are well known in the art. Tackifiers contemplated include SYLVATEC, ZONAREZ and FORAL which are available from Arizona Chemical and Hercules, Inc.

As previously stated, the adhesive compound is a hot melt adhesive. A preferred composition has a feasible temperature range for working with the hot melt adhesive in the range of about 275° F. to 350° F. The preferred temperature range for compounding and coating with the adhesive is 290° F. to 320° F.

Applicants have found that the addition of a heat stable antimicrobial agent to the above adhesive composition results in an effective antimicrobial adhesive composition. In particular, Applicants have found that the addition of diiodomethyl-p-tolylsulfone to the above adhesive composition results in an effective antimicrobial adhesive which retains desirable properties during use and application at 275° F. to about 350° F. A preferred loading of antimicrobial agent to the adhesive is in the range of about 0.1% to about 2% by weight. A preferred loading is about 0.2% by weight to about 0.6% by weight of diiodomethyl-p-tolylsulfone to adhesive. The resulting heat stable antimicrobial containing adhesive is 100% solids and eliminates the need for use of a solvent and the requisite evaporation of such solvent. The hot melt adhesive can also be ethylene oxide sterilized under heat stress or radiation sterilized without loss of effectiveness of the antimicrobial.

A preferred source of diiodomethyl-p-tolylsulfone is AMICAL 48, available from Angus Chemical Company.

The antimicrobial containing adhesive composition of the present invention is manufactured by mixing thoroughly at elevated temperature the acrylic polymers and tackifiers. A temperature of about 250° F. to about 260° F. has been found to be adequate. Once mixed, the polymers and tackifiers are heated to 310° F. to 350° F. with continued mixing until uniform, followed by cooling to 290° F. to 295° F. The diiodomethyl-p-tolylsulfone is then added to the polymer and mixed until uniform. The resultant composition is packaged and cooled for subsequent hot melt applications.

As detailed below, the antimicrobial adhesive of the present invention was shown to be effective against a wide variety of microorganisms. The antimicrobial activity was determined by using a series of zone of inhibition tests, as are well known in the art. The effective release of antimicrobial from the adhesive is estimated from the measurement of a zone of inhibition (an area of inoculated plate where organisms do not grow) surrounding the sample.

The adhesive utilized for the tests included 2% diiodomethyl-p-tolylsulfone homogeneously dispersed as detailed above in an adhesive composition. The adhesive composition included 17% low molecular weight acrylic polymer (HRJ-4326 from Schenectady International, Inc.) and 67% medium molecular weight polymer (HRJ-10127 from Schenectady International, Inc.) along with 16% FLORAL 105 synthetic resin from Hercules, Inc. as a tackifier. The adhesive composition was prepared as detailed above. The adhesive composition was then melted and applied to a substrate layer 14 in a thin coating (approximately 0.05 mm in thickness). The substrate was a co-polyester surgical drape material available from DuPont under the tradename HYTREL. The coated substrate 14 was cut to 6.0 mm disks for use in testing.

Adhesive coated disks were then exposed to microorganisms using the following procedure:

1. A microbial suspension containing=1.0.times.10.sup.8 organisms per ml in TSB was compared to the turbidity of a 0.5 MacFarland Standard.

2. A sterile swab was dipped into the culture suspension. The swab was rotated several times, pressing firmly on the inside wall of the tube above the fluid level. This removed excess inoculum from the swab.

3. The surface of a Mueller Hinton agar plate was inoculated by streaking the swab over the entire sterile agar surface. This streaking procedure was repeated two more times, rotating the plate approximately 60° each time to ensure an even distribution of inoculum.

4. The paper liner was removed from each 6 mm adhesive coated disc and the film was aseptically placed adhesive side down on the surface of the inoculated agar plate. Control samples were handled identically.

5. Immediately following the addition of the discs, the Mueller Hinton agar plates were placed in ambient air at 35-37° for 18-24 hours. Following incubation, the zones of inhibition surrounding the discs were measured. When no zone was observed, the disc was aseptically removed and the area beneath the disc was evaluated for growth of the test organism. The tests were repeated two or three times, using relevant microorganisms. Experimental results are presented in the table below, reported as the average diameter zone of inhibition surrounding/under 6.0 mm samples. A 6.0 mm zone of inhibition indicates no growth of the test organism beneath the 6.0 mm test discs, while larger zones indicate effective antimicrobial activity at a distance from the disc.

TABLE I

| Test Organism | Zone of Inhibition |
|---|---|
| Staphylococcus aureus | 12.0 mm |
| (ATCC 6538) | |
| Escherichia coli | 6.0 mm |
| (ATCC 11229) | |
| Pseudomonas aeruginosa | 6.0 mm |
| (ATCC 15442) | |
| Klebsiella pneumoniae | 7.0 mm |
| (ATCC 4352) | |
| Pseudomonas cepacia | 6.0 mm |
| (ATCC 25416) | |
| Enterobacter cloacae | 6.0 mm |
| (ATCC 13047) | |
| Serratia marcescens | 6.5 mm |
| (ATCC 14746) | |
| Streptococcus pyogenes | 10.5 mm |
| (ATCC 19615) | |
| Enterococcus faecalis- | 9.5 mm |
| Vancomycin Resistant | |
| (ATCC 51299) | |
| Candida albicans | 33.5 mm |
| (ATCC 10231) | |
| Bacillus subtilis | 9.2 mm |
| (ATCC 19659) | |

These results indicate that the present adhesive is effective in inhibiting these eleven relevant organisms, even after hot melt processing and ethylene oxide sterilization.

As earlier suggested, one form of subject antimicrobial adhesive composition or system of the subject invention may be fairly characterized as being solvent based, such system not requiring a solution or concentrate of the antimicrobial agent or the drying out of the solvent following the spreading or coating on a substrate. As the disclosed antimicrobial adhesive system utilizes antimicrobial agent in the form of a solid powder, it is readily, and directly compounded into the pressure sensitive acrylic adhesive, then, spread or coated onto a substrate (e.g., a surgical drape) without the drying requirement of the solid form of the adhesive as has been the typical and widespread practice. No additional solvent from agent is added. The subject system does not require an emulsion of the antimicrobial agent and the removal of excess solvent. Such composition formulation thereby simplifies a substrate coating process, and further allows for a more consistent, predictable, and effective result.

Further with respect to the antimicrobial agent of the subject system, the use of elemental iodine or an iodide salt in a solvent solution to provide an iodine active kill mechanism is avoided, instead the subject active antimicrobial agent is characterized by a molecule containing two iodides stabilized by the larger tolyl-phenol based group. The iodides of such molecule are harder to remove, thereby providing, among other advantages: a more heat stable and less water soluble antimicrobial agent; easier manufacture due as no special packaging and handling is required; less volatility when performing ETO sterilized; and, reduced susceptibility to leaching of the adhesive in wet environments.

The subject antimicrobial adhesive composition or system, especially suited for skin contact applications, generally includes an acrylic polymer, and an effective amount of diiodomethyl-p-tolylsufone dispersed throughout the acrylic polymer. The acrylic polymer suitably comprises a mixture of acrylic resin solutions, more particularly, self-curing and non self-curing acrylic resin solutions, the non self-curing acrylic resin solution preferably present in the mixture to a greater extent than the self-curing acrylic resin solution.

The majority acrylic resin solution of the mixture of acrylic resin solutions of the preferred antimicrobial adhesive composition general includes ethyl acetate, toluene, and about 40-45 wt % solids, more particularly, about 83 parts by weight ethyl acetate, about 17 parts by weight toluene, and about 40-42 wt % solids. Such acrylic resin solution is commercial available under the tradename Gelva® Multipolymer Solution 788.

The minority acrylic resin solution of the mixture of acrylic resin solutions of the preferred antimicrobial adhesive composition general includes ethyl acetate, ethanol, toluene, and about 30-35 wt % solids, more particularly, about 48 parts by weight ethyl acetate, about 40 parts by weight ethanol, about 12 parts by weight toluene, and about 31-34 wt % solids. Such acrylic resin solution is commercial available under the tradename Gelva® Multipolymer Solution 737.

The antimicrobial agent of the subject antimicrobial adhesive composition or system, as previously noted, comprises a diiodomethyl-p-tolylsulfone, e.g., AMICAL 48, available from Angus Chemical Company. A preferred antimicrobial adhesive composition, and one subject to testing subsequently discussed, is characterized as follows:

| Wt % | Constituent |
|---|---|
| 89.67 | Gelva ® solution 788 |
| 10.00 | Gelva ® solution 737 |
| 0.33 | AMICAL 48 (5% in ethyl acetate) |
| 0.0245 | DC Yellow 11 (0.3% in toluene) |
| 0.0205 | MX 643 (0.5% in toluene) |

As to efficacy testing, an exemplary sheet sample was produced comprising a cover sheet, 1.5 mils of acrylic PSA, bioflects 235-01, and a casting sheet. The solid content of aforementioned 90%/10% solution is about 41.25% which equates to 0.8% solids on solids of antimicrobial. The efficacy results of the subject system, namely zone of inhibition and log reduction effectiveness testing, and a discussion thereof, generally follows.

With regard to the zone of inhibition testing, the protocol associated therewith identifies the antimicrobial surfaces by placing a 6.5 mm disk of the test material on agar seeded with one cultured organism incubated and evaluated for the size (diameter) under the disk free of organisms. This zone may extend beyond the size of the disk depending on the level of antimicrobial activity. Antibiotics and skin prep solutions will have much larger zones than antimicrobial adhesives due to the high available concentration of the antimicrobial agent. Antimicrobial adhesives are predominantly effective on contact, and therefore are effective under the disk but not much beyond the disk's border. Three different organisms were tested with the test article, namely the sheet samples heretofore described, with 1.5 mils of the preferred solvent based antimicrobial adhesive system, gentamicin antibiotic positive control, and a plastic disk negative control.

With reference now to Tables IIA & IIB, the test article killed on contact under the disk with a zone of 6.5 mm. The positive control showed significant kill, and the negative control showed no zone or kill under the disk. These results are consistent with the hot melt composition characterized by the subject diiodo-sulfone antimicrobial agent.

With regard to Table IIA, a stock suspension of Pseudomonas Aeruginosa (ATC #9027) was diluted with sodium chloride, the concentration being adjusted to 1×10⁸ CFU/ML using 0.5 McFarland standard. Zones of inhibition were measured, and are reported in millimeters.

With regard to the results of Table IIB, stock suspensions of Pseudomonas Aeruginosa, Escherichia coli (ATC #8739) and Staphylococcus aureus (ATCC #6538) were diluted with the concentration adjusted to 1×10⁸ CFU/ML using 0.5 McFarland standard. Zones of inhibition were measured, and are reported in millimeters.

Log reduction effectiveness testing was conducted on two occasions, Tables IIIA-IIIC, and Tables IVA & IVB being associated with results for the separate test occasions. Log reduction of inoculated antimicrobial materials, more particularly the protocol associated therewith, identifies the time log reduction of inoculated organisms on test article surfaces. Five different test organisms were tested with the subject solvent based antimicrobial adhesive system as applied to an incised film, and a positive control, namely, the heretofore described and disclosed solventless hot melt as applied to an incise film.

The first study, (i.e., that relating to Tables III), indicate that the subject solvent based antimicrobial adhesive system was at least equivalent for three of the organisms tested. However, E. coli and E. faecium required the use a different dilution factor for the inoculum. In the second, later study (i.e., that associated with Tables IV), the subject organisms were tested at a higher dilution factor (0.01 ml), as was the fungus C. albicans. The results indicate that the subject solvent based antimicrobial adhesive is at least equivalent to the solventless hot melt previously described herein.

New characteristics and advantages of the invention covered by this document have been set forth in the foregoing description. It will be understood, however, that this disclosure is, in many respects, only illustrative. Changes may be made in details, particularly in matters of shape, size, and arrangement of parts, without exceeding the scope of the invention. The scope of the invention is, of course, defined in the language in which the appended claims are expressed.

What is claimed is:

1. An adhesive composition having antimicrobial properties for skin contact applications comprising:
a. an acrylic polymer; and,
b. an effective amount of diiodomethyl-p-tolylsufone dispersed throughout said acrylic polymer.

2. The adhesive composition of claim 1 wherein said acrylic polymer comprises a mixture of acrylic resin solutions.

3. The adhesive composition of claim 2 wherein a concentration of diiodomethyl-p-tolylsulfone in said acrylic polymer is about 0.1% to about 2% by weight.

4. The adhesive composition of claim 3 wherein a concentration of diiodomethyl-p-tolylsulfone in said acrylic polymer is about 0.1% to about 1% by weight.

5. The adhesive composition of claim 4 wherein a concentration of diiodomethyl-p-tolylsulfone in said acrylic polymer is about 0.1% to about 0.5% by weight.

6. The adhesive composition of claim 2 wherein said mixture of acrylic resin solutions includes a non self-curing acrylic resin solution.

7. The adhesive composition of claim 6 wherein said mixture of acrylic resin solutions includes a self-curing acrylic resin solution.

8. The adhesive composition of claim 7 wherein a concentration of diiodomethyl-p-tolylsulfone in said acrylic polymer is about 0.1% to about 2% by weight.

9. The adhesive composition of claim 8 wherein a concentration of diiodomethyl-p-tolylsulfone in said acrylic polymer is about 0.1% to about 1% by weight.

10. The adhesive composition of claim 9 wherein a concentration of diiodomethyl-p-tolylsulfone in said acrylic polymer is about 0.1% to about 0.5% by weight.

11. The adhesive composition of claim 8 wherein said non self-curing acrylic resin solution comprises a majority of said mixture of acrylic resin solutions.

12. The adhesive composition of claim 11 wherein a weight based ratio of said non self-curing acrylic resin solution to said self-curing resin solution for said mixture of acrylic resin solutions is between about 7 and 10.

13. The adhesive composition of claim 12 wherein said non self-curing acrylic resin solution of said mixture of acrylic resin solutions comprises about 40-45 weight percent solids.

14. The adhesive composition of claim 13 wherein said self-curing acrylic resin solution of said mixture of acrylic resin solutions comprises about 30-35 weight percent solids.

15. An antimicrobial adhesive system not requiring an emulsion of an antimicrobial agent thereof and the removal of excess solvent, said system comprising an effective amount of diiodomethyl-p-tolylsufone dispersed throughout an acrylic polymer.

16. A surgical drape comprising a sheet of polymeric substrate and a coating of an adhesive composition overlying said polymeric substrate, said adhesive composition comprising an acrylic polymer having an effective amount of diiodomethyl-p-tolylsulfone dispersed therein.
